(19) United States
(12) Patent Application Publication
(10) Pub. No.: US 2014/0349947 A1
(43) Pub. Date: Nov. 27, 2014

(54) HAIR GROWTH AGENT (EMBODIMENTS) AND METHOD FOR HAIR REGROWTH

(71) Applicant: Aleksey Valentinovich Odintsov, Perm (RU)

(72) Inventor: Aleksey Valentinovich Odintsov, Perm (RU)

(21) Appl. No.: 14/346,970

(22) PCT Filed: Feb. 5, 2013
(86) PCT No.: PCT/RU2013/000080

§ 371 Date: Mar. 25, 2014

(30) Foreign Application Priority Data
Feb. 7, 2012 (RU) 2012103974

(51) Int. Cl. A61K 8/73; A61K 8/34; A61K 8/81; A61K 8/64; A61K 8/42; A61K 8/97; A61Q 7/00; A61K 8/41; A61K 8/49

(52) U.S. Cl. CPC A61K 8/735; A61K 8/41; A61K 8/34; A61K 8/8147; A61K 8/494; A61K 8/42; A61K 8/97; A61Q 7/00; A61K 8/64
USPC 514/20.7

(57) ABSTRACT

The invention relates to the field of cosmetology, in particular to the hair regrowth, to the compositions of sodium salts of unfractionated heparins, to the lipid and peptide nanocomplexes for external use, to the methods for inducing and/or stimulating hair growth and/or reducing loss of hair using these compositions in the area of hair loss.

### FIELD OF THE INVENTION

The invention relates to the field of cosmetology, in particular to the hair regrowth, to the compositions of sodium salts of unfractionated heparins, to the lipid and peptide nanocomplexes for external use, to the methods for inducing and/or stimulating hair growth and/or reducing loss of hair using these compositions in the area of hair loss.

### BACKGROUND OF THE INVENTION

“Method and compositions for promoting hair growth” is known from the patent 3.0 literature: International Application No.: PCT/US2011/040470 (published by WIPO on 22, Dec. 2011), IPC A61K 8/73, the applicant is Momenta Pharmaceuticals, Inc. (US).

The method for hair regrowth provides topical application of a low molecular weight heparin (which is hereinafter referred to as LMWH) to the required part of a body once a day for 3, 4, 12 weeks or longer, e.g. for an indefinite term. The authors described a gel-cream composition per 100 g:

| | | |
|---|---|---|
| Sodium LMWH | 0.2 | g |
| Lutrol E-400 | 15 | g |
| Liquid paraffin | 10 | g |
| Lutrol F-127 | 23 | g |
| Water up to | 100 | |

This method and the composition of the gel-cream are taken as a prototype for both proposed method for hair regrowth and embodiments of compositions for drugs.

Disadvantages of the prototype are:
1) no data on the use of gel-cream in mice and humans, only assumptions are stated by the authors,
2) lack of results of parenteral and topical application of low molecular weight heparins for the evaluation of their effectiveness in the hair regrowth from alopecia in humans (including androgenic alopecia),
3) a reference to the parenteral route of administration of the preparation per os is inadmissible, since as consequence a systemic effect will be developed, that will be not only a side effect for hair regrowth from alopecia, but it will also be the development of serious complications, as the process of restoration of human hair follicles is long-lasting (up to 2 years, and sometimes up to 3 years to achieve a pronounced effect depending on the area of hair loss and duration of the process). Contraindications and side effects of long-term application of heparins, especially through parenteral routes of administration, are well known in the world literature.

### SUMMARY OF THE INVENTION

The object of the invention is to provide compositions and a method for hair regrowth free from the disadvantages of the prototype.

The objective is solved by the features specified in the claims:

According to the first embodiment such a gel composition for hair regrowth or preventing loss of hair comprises:

0.08-0.8% sodium salts of unfractionated heparin,
0.15-3% lipid nanocomplex emulsion,
0.5-2% thickener,
0.1-0.2% emulsifier,
0.03-1.3% neutralizing agent,
0.01-0.03% preservative, and
the remainder being water.

According to the invention, the composition comprises triethanolamine as a neutralizing agent.

According to the invention, the pharmaceutically acceptable solvent in the composition is selected from the group consisting of water and polyols. The polyol is a glycol. Glycol is selected from the group consisting of propylene glycol, dipropylene glycol, hexylene glycol, butylene glycol, PEG 200, PEG 400 and glycerol.

According to the invention, non-carbomeric thickener in the composition is selected from the group consisting of organic thickeners and inorganic thickeners. Non-carbomeric thickener is an organic thickener which is a polymer. The polymer is selected from the group consisting of starches, resins (gums), pectin, casein, gelatin, phycocolloids and synthetic polymers. The polymer is selected from the group consisting of alginates and salts and derivatives thereof, acacia gum, carrageen, guar gum, karaya gum, locust bean gum, tragacanth gum, xanthan gum, hyaluronic acid and salts thereof and polydextrose. Or the polymer is selected from the group consisting of crosslinked acrylic acid copolymers, dimethicone copolyols, acrylic/acrylate copolymers, polyacrylamide, poly(ethylene-co-sodium acrylate), poly(acrylamide-co-sodium acrylate), poly[(sodium acrylate)-co-(vinyl alcohol)], sodium polymethacrylate, sodium polystyrene sulfonate, povidone and derivatives thereof, polyquatenuum compounds, polyvinyl alcohol, polyethylene oxide and poloxamers. The polymer is a crosslinked copolymer of acrylic acid. The crosslinked copolymer of acrylic acid is acrylate/C₁₀₋₃₀ alkyl acrylate crosspolymer.

According to the invention, a crosslinked homopolymer of acrylic acid is used as a thickener in the composition.

The crosslinked homopolymer of acrylic acid is selected from the group consisting of Carbopol 934, Carbopol 940, Carbopol 980, Carbopol 981 and Carbopol® Ultiez™ 10.

According to the invention, the auxiliary active agents in the composition include emollients and hair growth stimulants such as allantoin and dexpanthenol along with sodium salts of unfractionated heparins and lipid nanocomplexes.

According to the second embodiment such a gel composition for hair regrowth or preventing loss of hair comprises:

0.08-0.8% sodium salts of unfractionated heparin,
0.15-3% lipid nanocomplex emulsion,
0.001-20% peptide nanocomplex solution,
0.05-3% thickener,
0.1-0.2% emulsifier,
0.03-1.3% neutralizing agent,
0.01-0.03% preservative, and
the remainder being water.

According to the invention, the components specified for the composition of the 1st embodiment are comprised as auxiliaries.

According to the third embodiment such a gel-cream composition for hair regrowth or preventing loss of hair comprises:

0.08-0.8% sodium salts of unfractionated heparin,
3-5% lipid nanocomplex emulsion,
0.05-3% thickener,
0.03-1.3% neutralizing agent,
0.01-0.03% preservative,
12-15% oils,
3-4% emulsifier, and
the remainder being water.

According to the invention, the composition comprises oils: mono-, polyunsaturated, saturated fatty acids. Oils are, for example, Kalahari melon oil, sasanqua oil.

According to the invention, the emulsifier in the composition is completely of plant origin. The emulsifier is Planta M.

According to the invention, the composition comprises the components specified for the invention according to the 1st embodiment as auxiliaries.

According to the fourth embodiment such a gel-cream composition for hair regrowth or preventing loss of hair comprises:

0.08-0.8% sodium salts of unfractionated heparin,
3-5% lipid nanocomplex emulsion,
0.001-20% peptide nanocomplex solution,
0.05-3% thickener,
0.03-1.3% neutralizing agent,
0.01-0.03% preservative,
12-15% oils,
3-4% emulsifier, and
the remainder being water.

According to the invention, the composition comprises the components specified for the 1st and 3rd embodiments as auxiliaries.

The objective is solved by the features specified in the claim 24, such as a method for hair regrowth or preventing loss of hair caused by alopecia comprising topical application of the compositions of claim 1, or claim 17, or claim 18, or claim 23 to the area of hair loss, said compositions being selected depending on the duration and the area of hair loss.

And also by the features specified in the claim 27, namely heparin or a salt thereof formulated into preparations for topical hair regrowth is applied daily for 3 months, and after 3-month course on alternate days to the sites of hair loss.

According to the invention, reasons for use of the method for hair regrowth from alopecia may be: androgenic alopecia, any forms of alopecia areata (circumscribed), anagen phase state without hair growth, traumatic alopecia, cicatrical alopecia, heat-induced alopecia, stress alopecia, alopecia induced by autoimmune disease (e.g. by discoid lupus erythematosus or chronic lupus erythematosus), disease-related alopecia (e.g. related with hypo- or hyperthyroidism, iron deficiency, zinc deficiency), drug-induced alopecia (e.g. induced by hormonal contraceptives, retinoids, beta-adrenergic blockers, interferon, antineoplastic agents, allopurinol, bromocriptine); effects of: granulosarcoid, radiation therapy, poisonings (bismuth, arsenic, gold, boric acid, thallium).

According to the invention, the method for hair regrowth comprises applying to the sites of hair loss of the abovementioned compositions once daily for a period of time required for a complete recovery, for at least 2, 6, 12, and 18 months or more.

According to the invention, the compositions described above are administered in a combination of the proper forms: gel, gel-cream.

The abovelisted set of essential features allows achieving such following technical result as increase in the range of means for hair regrowth (e.g., androgenic alopecia) without side effects.

The compositions according to the invention preferably comprise sodium salts of unfractionated heparin, and in the preferred embodiment, lipid and peptide nanosomal complexes (hereinafter referred to as nanocomplexes).

### 1. Sodium Salts of Unfractionated Heparins

Their implication is to stimulate hair growth.

It is known that the anticoagulant effect of sodium salts of unfractionated heparin occurs when it is administered intravenously, intramuscularly, subcutaneously.

At that, in the composition of ointments and gels the dose of sodium salts of unfractionated heparin up to 1,000 IU/g does not have a systemic effect and does not affect the bleeding time. (H. Krapfenbauer. Experimental animal demonstration of the thrombolytic effect of heparin ointment with new ingredients. Wien Med Wochenschr 1965 May 15; 115: 417—8).

After application of ointment or gel, sodium salts of unfractionated heparin are distributed in the upper layers of skin, in which up to 50% of preparation can be deposited. This determines frequency and amount of the preparation per day. The effects of heparin in tissues after a single skin application last up to 8 hours.

Comparison of low molecular weight and unfractionated heparins is shown in Table 1.

Any systemic effect when applying sodium salts of unfractionated heparin and low molecular weight heparins for hair regrowth from alopecia should be considered as a complication.

TABLE 1
Comparison of the active substance according to the prototype

| | | Low molecular weight |
|---|---|---|
| | Unfractionated heparin | heparin |
| Molecular weight | from 5 to 40 kD | from 2.5 to 6.5 kD |
| Bioavailability | low | high |
| Half-life | 1* | 2-4 times longer |

Note:
*- [symbol] - conditionally, since the time depends on the mode of administration. Data are given from the web site http://www.rinj.ru/articles_2641.htm

Conclusion: The abovementioned differences are essential in hair regrowth, as they affect the dosage of active substances.

### 2. Lipid Nanocomplexes

During the hair regrowth from alopecia with preparations of sodium salts of unfractionated heparin, temporary suppression of sebaceous glands activity in the areas of hair loss is found, which leads to local infectious diseases of a skin, changes in the structure of the hair.

To eliminate this side effect, lipid nanocomplexes were successfully added to the complex of preparation for hair regrowth from alopecia as a second active substance.

Lipid nanocomplexes have a protective effect on sebaceous glands.

The lipid nanocomplex used for hair regrowth is the nanoemulsion Nano LPD'S Multivitamin (manufacturer: Infinitec Activos, Spain—infinitec-activos.com, cosmetics-line.ru/file/63.pdf.).

Separate positive effect of lipid nanoemulsions on hair growth has not been described: WIPO patent applications No.: PCT/BR2005/000222, 1020020014436.

In the compositions according to the invention, betaine, acetylcholine, choline, glycerophosphocholine, phosphatidyl choline, lysophosphatidyl choline, carnitine, acyl carnitine or sphingomyelin, either separately or mixed with each other, and/or their derivatives can be used in the lipid nanocomplexes.

The phospholipid composition according to the invention is organized preferably in nanosomal emulsions with size of particles from 20 nm to 50 nm represented by one layer of membrane.

It is particularly preferred if these compounds are present in a concentration from 0.0001% to 8% by weight, preferably from 0.1% by weight to 5% by weight, in terms of the total weight of the composition in each case.

In the compositions, triglycerides or mixtures thereof can be used, in which the fatty acids with chain length of C₈-C₁₆ are esterified with glycerol. Triglycerides with fatty acids with chain length of C₈-C₁₂ are particularly preferred. Products of this type are marketed under different names (e.g. Myritol 312 and 318, or Miglylol 810 and 812).

In the compositions, natural or synthetic triglycerides can be used including glyceryl esters and derivative mono-, di- and triglycerides, based on Cβ-Ciβ of fatty acids, modified by reaction with other alcohols (caprylic/capric triglycerides, wheat germ glycerides, etc.), polyglyceryl fatty acid esters (polyglyceryl-n, such as polyglyceryl-4 caprate, ceramides, polyglyceryl-2 isostearate, etc.) or castor oil, hydrogenated vegetable oil, sweet almond oil, wheat germ oil, sesame oil, hydrogenated cottonseed oil, coconut oil, avocado oil, peanut oil, rapeseed oil, corn oil, hydrogenated castor oil, shea butter, cocoa butter, soybean oil, mink oil, sunflower oil, safflower oil, macadamia oil, olive oil, hydrogenated fat, apricot oil, hazelnut oil, borage seed oil, fish oil, etc.

Emulsifying systems for nanoemulsions may comprise such components as ceteareth-20, ceteareth-12, glyceryl stearate, cetearyl alcohol and cetyl palmitate, present in Emulgin B2, Emulgade® SE, or ready-to-use nanoemulsion formulations can be used, such as BF200.

### 3. Peptide Nanocomplexes

During the hair regrowth from alopecia in patients with long-term 5-year process delayed pigmentation of newly grown hair was revealed.

In order to accelerate the hair pigmentation, peptide nanocomplexes were administered.

The peptide composition according to the invention is organized preferably in nanosomal solutions with particle size up to 100 nm.

Peptide nanocomplex may be a combination of: tridecapeptide, acetyl decapeptide-3 (Rejuline), acetyl hexapeptide-8, acetyl octapeptide. Peptide nanocomplexes can be combined with simple peptides such as: oligopeptide-20 (CG-IDP5), oligopeptide-24 (CG-EDP3).

There is no evidence of hair growth during the hair regrowth with peptide nanocomplexes as modulators in similar WIPO patent applications Nos: 10171854, 06812204, 06812203, PCT/KR2006/004352, PCT/KR2007/004405, PCT/KR2007/004895.

As used herein, the term “peptide” refers to a linear molecule, which is formed by combining amino acid residues via peptide bonds.

Peptide nanocomplexes according to the experiment show excellent skin permeability due to their low molecular weight thereby promoting hair growth, skin moisture increase, activating blood circulation of the hair roots in scalp and maintaining anagen phase of the hair growth cycle.

### EMBODIMENTS OF THE INVENTION

The invention is further described by the following examples. For reference only: 1 IU of heparin sodium=0.0077 mg (Mashkovskiy M. D. Lekarstvennyy sredstva. V dvukh chastyakh. Ch. II—12 izd., pererab. i dop.—M.: Meditsyna, 1993.—688 s.).

### Example 1

Gel preparation falling within the scope of present invention and comprising the active substances: heparin sodium—0.8%, lipid nanocomplex emulsion—3%; the auxiliaries: thickener—2%, emulsifier—0.2%, neutralizing agent—1.3%, preservative—0.03%, solvent (water)—q.s. up to 100%.

### Part I Solution

| | | |
|---|---|---|
| Heparin sodium | 7.7 | mg |
| Nano LPD'S Multivitamin | 30 | mg |
| Purified water USP | 400 | mg |
| Part II Dispersion | | |
| Carbopol ® 940 | 20 | mg |
| Purified water USP | 558 | mg |
| Alcohol USP | 0.5 | mg |
| Part III Neutralizing agent | | |
| Triethanolamine | 13.5 | mg |
| Part IV Preservative | | |
| Methyl parahydroxybenzoate | 0.3 | mg |

### Preparation Technique

Water and heparin sodium are mixed in said proportions to dissolve the sodium salts of heparin, and the resulting solution is mixed with nanoemulsion Nano LPD'S Multivitamin. Carbopol® 940, alcohol and water from part II are mixed, neutralizing agent from part III is added and mixed to obtain a homogeneous mass.

Solution of heparin sodium is gradually added to the solution prepared by mixing parts II and III. Preservative is added to the resulting mixture of substances after all the others.

### Example 2

Gel preparation falling within the scope of present invention and comprising the active substances: heparin sodium—0.4%, lipid nanocomplex emulsion—1%; the auxiliaries: thickener—1%, emulsifier—0.15%, neutralizing agent—0.6%, preservative—0.02%, solvent (water)—q.s. up to 100%.

### Example 3

Gel preparation falling within the scope of present invention and comprising the active substances: heparin sodium—0.08%, lipid nanocomplex emulsion—0.15%; the auxiliaries: thickener—0.5%, emulsifier—0.1%, neutralizing agent—0.03%, preservative—0.01%, solvent (water)—q.s. up to 100%.

Examples 1, 2, 3 illustrate the composition according to the first embodiment.

### Example 4

Gel preparation falling within the scope of present invention and comprising the active substances: heparin sodium—0.8%, lipid nanocomplex emulsion—3%, peptide nanocomplex solution—20%; the auxiliaries: thickener—2%, emulsifier—0.2%, neutralizing agent—1.3%, preservative—0.03%, solvent (water)—q.s. up to 100%.

### Example 5

Gel preparation falling within the scope of present invention and comprising the active substances: heparin sodium—0.4%, lipid nanocomplex emulsion—1%, peptide nanocomplex solution—10%; the auxiliaries: thickener—1%, emulsifier—0.15%, neutralizing agent—0.6%, preservative—0.02%, solvent (water)—q.s. up to 100%.

### Example 6

Gel preparation falling within the scope of present invention and comprising the active substances: heparin sodium—0.08%, lipid nanocomplex emulsion—0.15%; peptide nanocomplex solution—0.001%, the auxiliaries: thickener—0.5%, emulsifier—0.1%, neutralizing agent—0.03%, preservative—0.01%, solvent (water)—q.s. up to 100%.

Examples 4, 5, 6 illustrate the invention according to the second embodiment.

### Example 7

Gel-cream comprising the active substances: heparin sodium—0.8%; lipid nanocomplex—Nano LPD'S Multivitamin—5%; fatty phase—Kalahari melon oil—9%, sasanqua oil—6%, Planta M emulsifier—4%; the auxiliaries in aqueous phase: thickener—2%, neutralizing agent—1.3%, preservative—0.03%, solvent (water)—q.s. up to 100%.

Active Substances:

| | | |
|---|---|---|
| Heparin sodium | 7.7 | mg |
| Nano LPD'S Multivitamin | 50 | mg |
| Purified water USP | 353.5 | mg |
| Aqueous phase: | | |
| Carbopol ® 940 | 30 | mg |
| Triethanolamine | 13.5 | mg |
| Methyl parahydroxybenzoate | 0.3 | mg |
| Purified water USP | 355 | mg |
| Fatty phase: | | |
| Kalahari melon oil | 90 | mg |
| Sasanqua oil | 60 | mg |
| Planta M | 40 | mg |

### Preparation Technique:

Preparation of the aqueous phase is performed as in the Example 1.

Preparation of the fatty phase: The required amount of oils and emulsifier are placed into a fireproof dish. Containers with fatty and aqueous phase are put on a water bath.

The oils are heated until the emulsifier is fully dissolved. The oil should become completely transparent.

While the phases are heated, the active substances are prepared. The active substances must be previously diluted in liquid. It is necessary to wait for a few minutes before the active substances are completely dissolved. Both containers must be removed from the water bath. The oil is poured into the aqueous phase (gel) and is mixed using a mixer. Fairly thick gel-cream will be obtained. Previously prepared active substances are added to the sufficiently cooled (warm) gel-cream. It should be mixed thoroughly. After liquid active substances are added the gel-cream became softer and reached its final consistence.

### Example 8

Gel-cream comprising the active substances: heparin sodium—0.4%; lipid nanocomplex—Nano LPD'S Multivitamin—4%; fatty phase—Kalahari melon oil—8%, sasanqua oil—6%, Planta M emulsifier—3.5%; the auxiliaries in aqueous phase: thickener—1%, neutralizing agent—0.6%, preservative—0.02%, solvent (water)—q.s. up to 100%.

### Example 9

Gel-cream comprising the active substances: heparin sodium—0.08%; lipid nanocomplex—Nano LPD'S Multivitamin—3%; fatty phase—Kalahari melon oil—7%, sasanqua oil—5%, Planta M emulsifier—3%; the auxiliaries in aqueous phase: thickener—0.5%, neutralizing agent—0.3%, preservative—0.01%, solvent (water)—q.s. up to 100%.

Examples 7, 8, 9 illustrate the invention according to the third embodiment.

### Example 10

Gel-cream comprising the active substances: heparin sodium—0.8%; lipid nanocomplex—Nano LPD'S Multivitamin—5%, peptide nanocomplex solution—20%; fatty phase—Kalahari melon oil—9%, sasanqua oil—6%, Planta M emulsifier—4%; the auxiliaries in aqueous phase: thickener—2%, neutralizing agent—1.3%, preservative—0.03%, solvent (water)—q.s. up to 100%.

### Example 11

Gel-cream comprising the active substances: heparin sodium—0.4%; lipid nanocomplex—Nano LPD'S Multivitamin—4%, peptide nanocomplex solution—10%; fatty phase—Kalahari melon oil—8%, sasanqua oil—6%, Planta M emulsifier—3.5%; the auxiliaries in aqueous phase: thickener—1%, neutralizing agent—0.6%, preservative—0.02%, solvent (water)—q.s. up to 100%.

### Example 12

Gel-cream comprising the active substances: heparin sodium—0.08%; lipid nanocomplex—Nano LPD'S Multivitamin—3%, peptide nanocomplex solution—0.001%; fatty phase—Kalahari melon oil—7%, sasanqua oil—5%, Planta M emulsifier—3%; the auxiliaries in aqueous phase: thickener—0.5%, neutralizing agent—0.3%, preservative—0.01%, solvent (water)—q.s. up to 100%.

Examples 10, 11, 12 illustrate the invention according to the fourth embodiment.

Pharmaceutical compositions for topical application may be in various forms including, for example, solutions, gels, suspensions, creams, etc. Improvements in absorption may be achieved when the compositions for topical application are in the form of solution or gel, i.e. if the active ingredient being sodium salts of unfractionated heparins are dissolved in the carrier contrary to suspension compositions for topical application, i.e. those, in which the active ingredient is merely suspended in the composition.

Solutions for topical application are not quite suitable for hair regrowth in the scalp, as they do not remain in place long enough for absorption of satisfactory amount of the drug. Variants of sodium salts of unfractionated heparins preparations were considered, like jelly comprising a drug substance, and ointments. These compositions may not be pharmaceutically very “elegant” and also may not be suitable for use as hair growth stimulating medicines, especially from the cosmetic point of view.

The term “pharmaceutically acceptable”, as used herein, refers to materials that are generally not toxic or harmful to a patient when used in the compositions of the invention, including topical application by the methods described herein. The term “patient”, as used herein, refers to animals, including mammals, preferably humans.

The terms “gel” and “gel composition”, as used herein, refer to colloidal compositions, which are preferably semiliquid systems that may consist of small inorganic particles or may comprise large organic molecules with interpenetrating liquid.

The term “non-gel” refers to compositions of the invention, which are not in the form of gels. Examples of non-gel compositions include, for example, emulsions, viscous solutions, etc. The term “viscous” (“thickened”), as used herein, refers to compositions, wherein the viscosity is increased to a higher value than viscosity of water at room temperature.

The term “emulsion”, as used herein, refers to a mixture of two or more liquids, which may be, for example, in the form of a continuous phase (dispersion medium) and the dispersion phase Emulsions may be, for example, in the form of creams or lotions and may include, for example, oil-in-water emulsions, water-in-oil emulsions, multilayer emulsions and micro- and nanoemulsions. The term “suspension”, as used herein, refers to a dispersion mixture of finely powdered particles floating (suspended) in a liquid.

The term “single-phase gel”, as used herein, refers to gels that may comprise organic macromolecules, which are homogeneously distributed throughout a liquid in such a way that no interphase boundary is observed between the dispersed macromolecules and the liquid. Single-phase gels may be prepared of synthetic macromolecules (e.g., acrylic acid polymers) or natural gums (e.g., tragacanth gum).

The viscosity of compositions of the invention may vary and depends, for example, on whether the compositions are gel compositions or non-gel compositions. In case of gels, viscosity of the compositions at room temperature may vary from more than about 4,000 centipoises to about 5 million centipoises, with all combinations and “subcombinations” of intervals and particular viscosity values being included within this interval. More preferably, the viscosity of gel compositions of the invention may be about 5,000 to 50,000 centipoises, with the viscosity values of about 6,000 to 25,000 centipoises being still more preferred. In the case of non-gel compositions the viscosity of these compositions at room temperature may be of about 6 to 4,000 centipoises, with all combinations and “subcombinations” of intervals and particular viscosity values being included within this interval. More preferably, the non-gel compositions of the invention may have the viscosity value of about 50 to 3,000 centipoises, with the viscosity values of about 100 to 2,000 centipoises being still more preferred.

Concentration of sodium salts of unfractionated heparins in the compositions of the invention may vary. Generally, heparin and sodium salts thereof may be present in the compositions of the invention in an amount from 0.08 to 0.8% and in all combinations and “subcombinations” of intervals and particular values within this interval. As used herein, the term “%” refers to weight %, unless indicated otherwise. Moreover, the total % (the total percentage) of components in the compositions of the invention may not exceed 100%. In preferred embodiments, the concentration of sodium salts of unfractionated heparins is more than 0.7%, with the concentrations of about 0.72%, about 0.74%, about 0.75%, and about 0.76% being more preferred. In yet more preferred embodiments of the invention, sodium salts of unfractionated heparins may be present in an amount of about 0.69%) or in an amount of about 0.7%, with the particularly preferred concentration being about 0.8%.

Recommended dosage of lipid nanocomplexes in the gel-cream (depending on required consistence of cosmetic product) is between 3 and 5%. In case of using thickeners and emulsion stabilizers (gelling agents such as Carbopol, various naturally occurring gums, etc.) when formulating, the recommended dosage of lipid nanocomplexes may be reduced to between 0.15 and 3%.

It is particularly preferable when peptide nanocomplexes are present in a gel, gel-cream at a concentration from 0.001% to 20% by weight.

In the compositions waxes can be used, including esters of long-chain acids and alcohols as well as compounds with wax-like property, such as carnauba wax, beeswax (white or yellow), lanolin wax, ozokerite, Japanese wax, paraffin, microcrystalline wax, ceresin, wax esters, synthetic wax, etc., or hydrophilic waxes.

It is found that desirable pharmaceutically acceptable characteristics may be obtained by adding water to the compositions of the invention. The term “pharmaceutically acceptable” as used herein means that compositions are preferably homogeneous by touch, non-oily and containing no solid impurities.

The amount of the solvent, i.e. water used in compositions of the invention, may vary and depends, for example, on the amount of sodium salt of unfractionated heparin used, other active substances, thickener, additives, etc. Preferably, the solvent may be used in the compositions of the invention in approximate amounts of between 51.87 and 99.13% and in all combinations and “subcombinations” of intervals and particular values within this interval.

Also in preferred form, the ratio of the solvent to the sodium salt of unfractionated heparin in the compositions of the invention is about 10:0.08. Ratios (proportions) as described herein represent weight/weight ratios (proportions).

According to preferred embodiments of the invention, higher viscosity values of the compositions of the invention may be obtained using thickeners. The term “thickener”, as used herein, refers to any of a number of common hydrophilic materials, which, when included into compositions of the invention, may act as viscosity modifying agents, emulsifiers, gelling agents, suspending agents and/or stabilizing agents. It is considered that due to such properties thickeners may contribute to stabilizing of the composition. If required, two or more thickeners can be used in compositions of the invention.

Suitable polymeric thickeners in the compositions of the invention include, for example, casein, gelatin, starch, gum, pectin, phycocolloids, and synthetic polymers. Examples of the abovementioned materials are salts of alginic acid and derivatives thereof, including, for example, sodium alginate and propylene glycol alginate, acacia gum, carrageen, guar gum, karaya gum, locust bean gum, tragacanth gum, xanthan gum, hyaluronic acid and salts thereof, such as, for example, sodium hyaluronate, gelatin and polydextrose.

Other polymeric thickeners, which may be used, include, for example, acrylic acid polymers, such as crosslinked acrylic acid interpolymers, polyacrylic acid and salts thereof, crosslinked acrylic acid homopolymers, crosslinked acrylic acid copolymers, acrylic/acrylate copolymers which are dimethicone copolyols, polyacrylamide, poly[(sodium acrylate)-co-(vinyl alcohol)], sodium polymethacrylate, sodium polystyrene sulfonate, poly(ethylene-co-sodium acrylate), poly(acrylamide-co-sodium acrylate), povidone and derivatives thereof, polyquaternium compounds, such as polyquaternium 10, polyvinyl alcohol, polyethylene oxide and poloxamers.

Any of the abovelisted thickeners may be used in the compositions of the invention. In some preferred embodiments, thickeners may be non-carbomeric thickeners. Non-carbomeric thickener is not a heterobiopolysaccharide or a cellulose derivative.

The term “carbomer”, as defined by The Cosmetic, Toiletry and Fragrance Association (CTFA) and as used herein, refers to synthetic high molecular weight crosslinked acrylic acid homopolymers. Examples of carbomers include, for example, Carbopol, such as Carbopol 934, Carbopol 940, Carbopol 980, Carbopol 981 and Carbopol® Ultrez™ 10, commercially manufactured by B.F. Goodrich (Cleveland, Ohio).

The term “non-carbomeric”, as used herein, refers to thickeners which are not carbomers.

Some carbomers are particularly applicable in compositions containing increased amounts of solvent, for example protic solvents, such as alcohols and polyols, and reduced amounts of water.

Such carbomers are referred to herein as “solvent-tolerant carbomers” and include such carbomers as, for example, Carbopol® Ultrez™ 10 and Carbopol® 934P, 940, 941, 980, and 981 (all are commercially manufactured by B.F. Goodrich).

Other thickeners may be acrylic acid copolymers, with the crosslinked acrylic acid copolymers being more preferred. Particularly preferred among these thickeners are acrylate/C₁₀₋₃₀ alkyl acrylate crosspolymers.

Examples of acrylate/C₁₀₋₃₀ alkyl acrylate crosspolymers, which may be suitable for use in the compositions of the invention, are polymeric emulsifiers Pemulen®, including Pemulen® TR1 and Pemulen® TR2.

In an alternative preferred embodiment of the invention the compositions of the invention may comprise inorganic thickeners. Suitable inorganic thickeners include, for example, bentonite, magnesium aluminum silicate and colloidal silica.

The amount of the thickener used in the compositions of the invention may vary and depends, for example, on the particular polymer and solvent used, specified viscosity of the final composition, etc. In general, the thickener may be used in amounts providing specified viscosity of the composition.

Preferably, the thickener may be used in an amount of about 1 to 3% and in combinations and “subcombinations” of intervals and particular amounts within this interval. More preferably, thickeners may be used in an amount of about 1 to 2%, with more preferred range of about 1 to 1.1%.

Moreover, the compositions of the invention may comprise one or more neutralizing agents, which may be used to adjust the pH. The term “neutralizing agent”, as used herein, refers to a material (a substance) that can be used to modify pH of the composition of the invention, for example, from acidic pH to more alkaline pH, or from alkaline (basic) pH to more acidic pH.

Components of the compositions of the invention, such as certain thickeners, may be acidic and preferably may be neutralized to achieve the desired viscosity.

Accordingly, neutralizing agents are preferably the substances, which can be used to modify pH of the compositions of the invention from acidic pH to more basic pH.

A wide range of neutralizing agents known to those skilled in the art and can be employed in practical use of the invention. Exemplary neutralizing agents include, for example, ammonium hydroxide, arginine, 2-amino-2-methyl-1-propanol (AMP-95® (Angus)), diethanolamine, triethanolamine, dimethanolamine, dibutanolamine, diisobutanolamine, tributanolamine, triisobutanolamine, tripropylamine, ethanolamine, PEG 15 cocamine, diisopropylamine, methylethanolamine, dipropylenetriamine, tromethamine, isopropylamine, ethylene diamine, triisopropanolamine, tetrahydroxypropyl ethylenediamine, trimethamine, 2-aminobutanol, aminoethyl propanediol, aminomethyl propanediol, aminomethyl propanol, sodium hydroxide, potassium hydroxide and mixtures thereof.

Preferably, the neutralizing agent is selected from triethanolamine, diethanolamine, tromethamolum and mixtures thereof. More preferred neutralizing agent is triethanolamine.

The amount of the neutralizing agent used in the compositions of the invention may vary and depends, for example, on the particular neutralizing agent and thickener used, amount of thickener to be neutralized, required pH, etc. Preferably, the neutralizing agent may be used in the compositions of the invention in amounts in the range of about 0.1 to 1.35% (and in all combinations and “subcombinations” of intervals and particular amounts within this interval), of total weight of the composition. More preferably, the neutralizing agent may be used in the compositions of the invention in an amount of about 1.2 to 1.35%. Yet more preferably, the neutralizing agent may be used in the compositions of the invention in an amount of about 1.35%. The amount of the neutralizing agent may also be expressed as a ratio of the thickener to the neutralizing agent. The ratios used herein are weight ratios (weight/weight). More preferred ratios of the thickener to the neutralizing agent are the ratios of about 1:1.3, with yet more preferred ratio being 1:1.35.

In addition to sodium salts of unfractionated heparin, lipid nanocomplexes, thickener, neutralizing agent and pharmaceutically acceptable solvent, the compositions of the invention may preferably comprise polar protic solvents. Preferably, the solvent is a hydroxyl compound, for example a compound containing at least one hydroxyl (OH) group. Alcohols (i.e., compounds containing one hydroxyl group) or polyols (i.e., compounds containing two or more hydroxyl groups), or mixtures of alcohols and/or polyols are preferred among the hydroxyl compounds. Examples of alcohols include ethanol, propanol and butanol. The term “ethanol”, as used herein, includes absolute alcohol, as well as “alcohol USP” and all denaturated forms of 95% ethanol. The term “propanol”, as used herein, refers to all isomeric forms, including n-propanol and isopropanol, and the term “butanol” refers to all isomeric forms, including, for example, isobutanol. Ethanol and propanol are preferred among these alcohols, with ethanol being more preferred.

Preferably, the polar solvent may be used in the compositions of the invention in approximate amounts of 0.05 to 1% and in all combinations and “subcombinations” of intervals and particular values within this interval. More preferably, the solvent is used in an amount of at least about 0.05%.

The compositions of the invention may be topically administered to the area (surface) of a patient to prevent hair loss or to promote hair regrowth.

Cosmetic compositions may be in the form of paste, cream or gel, serum, aerosol, foam, elixir, they may include animal and vegetable fats, starch, tragacanth, cellulose derivatives, polyethylene glycols, silicones, bentonites, silica, talc, zinc oxide or mixtures of these substances.

In the formulation of powder or spray, the composition may comprise lactose, talc, silica, aluminum hydroxide, calcium silicate, polyamide powder and mixtures of these substances. The spray may additionally comprise, for example, chlorofluorohydrocarbons, propane/butane or dimethyl ether.

The emulsion composition may comprise solvents, a solvent and an emulsifier, for example, water, ethanol, isopropanol, ethyl carbonate, ethyl acetate, benzyl alcohol, benzyl benzoate, propylene glycol, 1,3-butylglycol, oils, glycerol, polyethylene glycol fatty acid esters, and sorbitan fatty acid esters.

The compositions described herein may also be in the form of shampoo, conditioner, no-rinse perfume hair mask, mousse, hair gel, hair spray optionally in combination with a dye and/or other hair care products for cleaning, styling, hair regrowth, conditioning, or in the form of hair dyes simultaneously with topical application of sodium salts of unfractionated heparins, lipid and peptide nanocomplexes as described herein.

A large number of methods can be used to prepare the compositions of the invention. In general, the compositions can be prepared by combining the components of compositions described herein at the temperature and during the time sufficient to preferably provide a pharmaceutically acceptable composition.

The term “combination (combining) (together)”, as used herein, means that all the components of the compositions can be combined and mixed approximately simultaneously.

Method for Hair Regrowth from Alopecia.

Hair regrowth was further accomplished in 3 groups of volunteer patients with duration of hair loss:

| | |
|---|---|
| 1. | from 6 months to year (3 persons), |
| 2. | 2-3 years (3 persons), |
| 3. | about 5 years (5 persons). |

### Examples of Hair Regrowth in Groups

### Group 1

### Example 1

### Patient C., 32 Years Old

Suffered from hair loss on the area of 10 cm² for 1 year. Localization of hair loss: fronto-temporal regions (5 cm² on each side). Period of hair regrowth: February-March 2009. Percentage of hair regrowth: 100%.

The compositions in hair regrowth: heparin sodium 1,000 IU/g (gel).

Side effects during hair regrowth: impaired skin barrier function.

Hair regrowth regimen: heparin sodium 1,000 IU/g (gel) topically with interruptions for 1-2 weeks.

Pigmentation of grown hair: restored simultaneously with the growth of hair.

### Group 2

### Example 2

### Patient B., 35 Years Old

Suffered from hair loss on the area of 25 cm² for 2 years. Localization of hair loss: area of the scalp.

Period of hair regrowth: September 2010-December 2011.

Percentage of hair regrowth: 90%.

The compositions in hair regrowth: heparin sodium 1,000 IU/g (gel), Nano LPD'S Multivitamin (http://www.acef.it/8080/acef/doc/Caralogo_Farmacia.pdf).

Side effects during hair regrowth: impaired skin barrier function prior to the including of lipid nanosomes to the course.

Hair regrowth regimen: heparin sodium 1,000 IU/g (gel) topically with interruptions for 1-2 weeks until the end of 2010, lipid nanosomes were included into the preparation composition in 2011.

Pigmentation of grown hair: delayed, but does not require correction as it is almost completed by the end of hair regrowth.

### Example 3

### Patient B., 37 Years Old

Suffered from hair loss on the area of 30 cm² for 3 years. Localization of hair loss: area of the scalp.

Period of hair regrowth: June 2010-December 2011.

Percentage of hair regrowth: 90%.

The compositions in hair regrowth: heparin sodium 1,000 IU/g (gel, gel-cream), Nano LPD'S Multivitamin.

Side effects during hair regrowth: impaired skin barrier function prior to the including of lipid nanosomes to the course.

Hair regrowth regimen: heparin sodium 1,000 IU/g (gel) topically with interruptions for 1-2 weeks until the end of 2010, without interruption in 2011 after the including of the lipid nanosomes into the formulation.

Due to the duration of hair loss and low regeneration rate, the composition was enriched by the gel-cream form comprising oils, including such containing the antioxidants.

Pigmentation of grown hair: delayed, but does not require correction as it is almost completed by the end of hair regrowth.

### Example 4

### Patient C., 38 Years Old

Suffered from hair loss on the area of 50 cm² for 3 years (rapidly progressing form). Localization of hair loss: area of the scalp.

Period of hair regrowth: January 2010-December 2011. Percentage of hair regrowth: 90%.

The compositions in hair regrowth: heparin sodium 1,000 IU/g (gel, gel-cream), Nano LPD'S Multivitamin, revitalizing gel for skin (peptide nanocomplex—DERM-16—(http://pcosmetic.m/catalog.php?filter=brand&name=dermaheal&folder=28).

Side effects during hair regrowth: impaired skin barrier function prior to the including of lipid nanosomes to the course.

Hair regrowth regimen heparin sodium 1,000 IU/g (gel) topically with interruptions for 1-2 weeks until the end of 2010, without interruption in 2011 after the including of the lipid nanosomes into the formulation. Due to the duration of hair loss and low regeneration rate, the composition was combined with the gel-cream form comprising oils, including such containing the antioxidants.

Pigmentation of grown hair: delayed, requires correction. Pigmentation of grown hair is delayed for 1 year.

While growing, hair was gaining color with delay, and then the DERM-16 preparation was added to the hair regrowth. In November 2011, acceleration of newly grown hair pigmentation by 25-30% was observed.

### Group 3

### Example 5

### patient C., 42 years old

Suffered from hair loss on the area of 50 cm² for 5 years (slowly progressing form). Localization of hair loss: area of the scalp. Period of hair regrowth: January 2009-December 2011. Percentage of hair regrowth: 90%.

The compositions in hair regrowth: heparin sodium 1,000 IU/g (gel), Nano LPD'S Multivitamin, revitalizing gel for skin (peptide nanocomplex—DERM-16).

Side effects during hair regrowth: impaired skin barrier function prior to the including of lipid nanosomes to the course.

Hair regrowth regimen: heparin sodium 1,000 IU/g (gel) topically with interruptions for 1-2 weeks until the end of 2010, without interruption in 2011 after the including of the lipid nanosomes into the formulation.

Slow progression of hair loss indirectly indicated the high regenerative ability of hair follicles and did not required addition of gel-cream to the hair regrowth.

Pigmentation of grown hair: delayed, requires correction. Pigmentation of grown hair is delayed for 1.5 year.

While growing, hair was gaining color with delay, and then the DERM-16 preparation was added to the hair regrowth in 2011. In November 2011, acceleration of newly grown hair pigmentation by 25-30%, hair growth acceleration by 20% was observed.

### Example 6

### Patient K., 35 Years Old

Suffered from hair loss on the area of more than 100 cm² for 5 years (rapidly progressing form). Localization of hair loss: frontal region and area of the scalp. Period of hair regrowth: hair regrowth continues from January 2009. Percentage of hair regrowth: 70%.

The compositions in hair regrowth: heparin sodium 1,000 IU/g (gel, gel-cream), Nano LPD'S Multivitamin, revitalizing face cream (peptide nanocomplex—DERM-17).

Side effects during hair regrowth: impaired skin barrier function prior to the including of lipid nanosomes to the course.

Hair regrowth regimen: heparin sodium 1,000 IU/g (gel) topically with interruptions for 1-2 weeks until the end of 2010, without interruption in 2011 after the including of the lipid nanosomes into the formulation.

Pigmentation of grown hair: delayed, requires correction.

Pigmentation of grown hair is delayed for 1.5 year. While growing, hair was gaining color with delay, and then the DERM-17 preparation was added to the hair regrowth in 2011 (http://pcosmetic.m/catalog.php?filter=brand&name=dermaheal&folder=28). In November 2011, acceleration of newly grown hair pigmentation by 25-30%, hair growth acceleration by 20% was observed.

### General Hair Regrowth Conditions:

Method for hair regrowth using the compositions consisted in applying the formulations to the sites of hair loss once a day.

### General Side Effects:

During the hair regrowth, hair growth was interchanged with no-growth periods with evidences of impaired skin barrier function.

Symptomatology caused by hair regrowth with sodium salts of unfractionated heparins is combined into sebaceous gland hyposecretion syndrome for the first time in the scope of the invention. Sebaceous gland hyposecretion syndrome is represented by hair growth impairment, skin and hair dryness, dysbacteriosis in the hair regrowth area exhibited by purulent pustules.

To eliminate the abovementioned complications of hair regrowth in 2009-2010, it was necessary to interrupt the course of hair regrowth for 1-2 weeks.

During periods of hair growth impairment a second active substance that eliminates these complications was sought. Among these preparations there were: Solcoseryl (gel), Actovegin (gel), topical antibacterial preparations; preparations with lipid (Nano LPD'S Multivitamin) and peptide (DERM-16, DERM-17) nanocomplexes.

Gel-cream is preferred for hair regrowth for the following reasons:

1) a possibility to include oils, emollients into the preparation composition,
2) a milder action during long-term hair regrowth,
3) a possibility to increase the nourishing and moisturizing agents, antioxidants in the composition,
4) maintaining absorption capacity of the active agents by aqueous phase,
5) rapidly progressing forms of hair loss.

Hair regrowth effectiveness in the group 3 is shown in Table 2, the selection of the form of preparation is shown in Table 3.

TABLE 2
Evaluating the effectiveness of hair regrowth from androgenic
alopecia depending on the dosage, base (gel, gel-cream, ointment)
of the preparation in the group 3.

| | | | 2011 | |
|---|---|---|---|---|
| | | | first half | second half |
| Compositions | 2009 | 2010 | of the year | of the year |
| Heparin sodium 1,000 IU/g | 50%* | 60% | 70% | 90% |
| gel (2 pers.) + compositions | | | | |
| of example 5 | | | | |
| Heparin sodium 1,000 IU/g | 30% | 40% | 50% | 70% |
| gel-cream (1 pers.) + | | | | |
| compositions of example 6 | | | | |
| Hepathrombin 50 gel (1 pers.) | 20% | 30% | 40% | 40% |
| Heparin ointment (1 pers.) | — | 5% | 10% | — |

Note:
*- Percentage of hair regrowth from the initial level of hair loss.

TABLE 3
Approximate selection of a preparation for hair regrowth from alopecia
(by the example of hair regrowth from androgenic alopecia)

| | | | | Composition | | | Approximate | |
|---|---|---|---|---|---|---|---|---|
| | Selection criterion | | | sodium | lipid | peptide | duration of | |
| | Duration of | Area of | | salts of | nano- | nano- | hair regrowth | |
| No. | hair loss | hair loss | Form | heparin | complexes | complexes | (to cosmetic effect) | |
| 1 | up to 1 year | 10-20 cm² | gel | + | + | | 2 | months |
| 2 | up to 2 years | 20-30 cm² | gel | + | + | | 1.5 | years |
| 3 | up to 3 years | 30-40 cm² | gel-cream | + | + | | 1.5 | years |
| 4 | up to 3 years | 40-50 cm² | gel-cream | + | + | + | 2 | years |
| 5 | up to 5 years | 40-50 cm² | gel | + | + | + | 2.5 | years |
| 6 | up to 5 years | >100 cm² | gel-cream | + | + | + | 3-3.5 | years |

Unexpectedly, the author who suffered from androgenic alopecia for 5 years, has found a new secondary use of sodium salts of unfractionated heparin for hair regrowth from alopecia by applying the compositions of heparin sodium to the sites of hair loss once a day (Russian Federation patent application No. 2011126646/15, filed Jun. 30, 2011, of the same applicant).

The preparations for topical administration used in the present method are as follows:

Heparin ointment. Composition: heparin 100 IU/g, heparin 2,500 U, Anaesthesinum 1 g, Nicotinic acid benzyl ester 0.02 g, Ointment base up to 25 g (Mashkovskiy M. D. Lekarstvennyy sredstva. 4. II—12 izd., pererab. i dop.—M.: Meditsyna, 1993.—688 s.).

Lioton (active substance is heparin). International Nonproprietary Name (INN): heparin sodium. Composition: 1 g of gel comprises heparin sodium salt 1,000 IU as active ingredient. Auxiliaries: methyl para-hydroxybenzoate, propyl parahydroxybenzoate, carbomer 940, ethyl alcohol 96%, neroli oil, lavender oil, triethanolamine, purified water. Direct anticoagulant for topical administration. (product label “LIOTON 1000” Marketing authorisation number Π No 01210701 of Jul. 14, 2006, Manufacturer “A. Menarini Industrie Farmaceutiche Riunite S.r.L.”). It is possible for other ointments and gels comprising heparin to be used in the method.

### Example 7

Hair regrowth from alopecia was conducted for 2 years and 4 months.

In the first year of hair regrowth there was daily topical application of Lioton gel on a scalp without controlling blood coagulation. There were no side effects as ulorrhagia, epistaxis, increased blood clotting time, traumatic haemorrhage.

Alopecia process had been existing for 5 years before the hair regrowth (from 2004 to January 2009). Result of hair regrowth was hair-covering restoration by 70% of initial hair loss on the scalp and frontal regions. The result was achieved during the first 2 months of hair regrowth. Supportive hair regrowth and monitoring the hair growth was conducted for the other 2 years. Hair growth regression was no longer observed. Due to the specific treatment, the regression of the process of alopecia to the state of almost 3 years before was achieved (to the level of hair loss in 2006). Thus, daily application of the gel is required to the sites of hair loss on the head (Lioton gel preparation) at any free time once a day for the first 3 months. After the 3-month course the application of the Lioton gel can be made alternate days. Complications: none.

### INDUSTRIAL APPLICABILITY

1. Hair regrowth from androgenic alopecia with the process lasting for 3-5 years is of long continuance.
2. Delivery of sodium salts of unfractionated heparin to the soft tissues is a dose-dependent effect.
3. Disadvantages of the ointment-based preparations are identified: low hair regrowth efficacy, signs of skin maceration, skin dryness.
4. Gel is the most effective form providing the penetration of active components into the skin layers.
5. Gel-cream is also the actual form for administration of active ingredients.
6. Sebaceous gland hyposecretion syndrome in the area of hair loss caused by application of sodium salts of unfractionated heparin (gel) for topical hair regrowth was identified as new clinical syndrome.
7. During hair regrowth from alopecia with sodium salts of unfractionated heparin (1 active substance, Russian Federation patent application No. 2011126646/15, filed Jun. 30, 2011, of the same applicant) an interruption is required to restore the function of sebaceous glands, which extends a course of hair regrowth and requires certain skills for reinitiation thereof.
8. Preventing sebaceous gland hyposecretion syndrome was achieved as a result: there were no complications related to the event of skin disbacteriosis within 6 months when lipid nanocomplexes were included as an additive (e.g., Nano LPD'S Multivitamin) into the formulations for hair regrowth from alopecia.
9. Peptide nanocomplexes were applied to accelerate hair growth and pigmentation.

The present description and examples are considered as material illustrating the invention, spirit of which and patent's scope being defined by the following claims, combination of essential features and equivalents thereof

What is claimed is:

1. A gel composition for hair regrowth or preventing loss of hair, comprising
0.08-0.8% sodium salt of unfractionated heparin,
0.15-3% lipid nanocomplex emulsion,
0.5-2% thickener,
0.1-0.2% emulsifier,
0.03-1.3% neutralizing agent,
0.01-0.03% preservative, and
the reminder being a solvent selected from the group consisting of water or polyol.

2. The composition of claim 1, comprising triethanolamine as a neutralizing agent.

3. The composition of claim 1, wherein the pharmaceutically acceptable solvent is selected from the group consisting of water and polyols.

4. The composition of claim 3, wherein the polyol is a glycol.

5. The composition of claim 4, wherein the glycol is selected from the group consisting of propylene glycol, dipropylene glycol, hexylene glycol, butylene glycol, PEG-200, PEG-400 and glycerol.

6. The composition of claim 1, wherein the solvent is water and present in the composition in an amount of at least about 92.7 to 99.28%.

7. The composition of claim 1, wherein the thickener is selected from the group consisting of non-carbomeric organic thickeners and non-carbomeric inorganic thickeners.

8. The composition of claim 7, wherein the non-carbomeric thickener is an organic thickener which is a polymer.

9. The composition of claim 8, wherein the polymer is selected from the group consisting of starches, resins (gums), pectin, casein, gelatin, phycocolloids and synthetic polymers.

10. The composition of claim 9, wherein the polymer is selected from the group consisting of alginates and salts and derivatives thereof, acacia gum, carrageen, guar gum, karaya gum, locust bean gum, tragacanth gum, xanthan gum, hyaluronic acid and salts thereof and polydextrose.

11. The composition of claim 10, wherein the polymer is selected from the group consisting of crosslinked acrylic acid copolymers, dimethicone copolyols, acrylic/acrylate copolymers, polyacrylamide, poly(ethylene-co-sodium acrylate), poly(acrylamide-co-sodium acrylate), poly[(sodium acrylate)-co-(vinyl alcohol)], sodium polymethacrylate, sodium polystyrene sulfonate, povidone and derivatives thereof, polyquaternium compounds, polyvinyl alcohol, polyethylene oxide and poloxamers.

12. The composition of claim 11, wherein the polymer is a crosslinked acrylic acid copolymer.

13. The composition of claim 12, wherein the crosslinked acrylic acid copolymer is acrylate/C₁₀₋₃₀ alkyl acrylate crosspolymer.

14. The composition of claim 1, wherein the thickener is a crosslinked acrylic acid homopolymer.

15. The composition of claim 14, wherein the crosslinked acrylic acid homopolymer is Carbopol 934, Carbopol 940, Carbopol 980, Carbopol 981 and Carbopol® Ultrez™ 10.

16. The composition of claim 1, wherein auxiliary active agents in the substance further include emollients and hair growth stimulants such as allantoin and dexpanthenol as well as unfractionated heparins and lipid nanocomplexes.

17. A gel composition for hair regrowth or preventing loss of hair, comprising:
0.08-0.8% sodium salt of unfractionated heparin,
0.15-3% lipid nanocomplex emulsion,
0.001-20% peptide nanocomplex solution,
0.05-3% thickener,
0.1-0.2% emulsifier,
0.03-1.3% neutralizing agent,
0.01-0.03% preservative, and the remainder being water.

18. A gel-cream composition for hair regrowth or preventing loss of hair, comprising:
0.08-0.8% sodium salt of unfractionated heparin,
3-5% lipid nanocomplex emulsion,
0.05-3% thickener,
0.03-1.3% neutralizing agent,
0.01-0.03% preservative,
12-15% oils,
3-4% emulsifier, and
the remainder being water.

19. The composition of claim 18, wherein the oils comprise mono-, polyunsaturated, saturated fatty acids.

20. The composition of claim 19, wherein the oils are Kalahari melon oil, sasanqua oil.

21. The composition of claim 18, wherein the emulsifier is completely of plant origin.

22. The composition of claim 21, wherein the emulsifier is Planta M.

23. A gel-cream composition for hair regrowth or preventing loss of hair, comprising:
0.08-0.8% sodium salt of unfractionated heparin,
3-5% lipid nanocomplex emulsion,
0.001-20% peptide nanocomplex solution,
0.05-3% thickener,
0.03-1.3% neutralizing agent,
0.01-0.03% preservative,
12-15% oils,
3-4% emulsifier, and
the remainder being water.

24. A method for hair regrowth or preventing loss of hair caused by alopecia, comprising topical application of the compositions of claim 1, or claim 17, or claim 18, or claim 23 to the area of hair loss, said compositions being selected depending on the duration and area of hair loss and applied to sites of hair loss once daily for a period of time required for full recovery for at least 2, 6, 12, and 18 or more months.

25. The method for hair regrowth of claim 24, wherein causes of the alopecia may be: androgenic alopecia, any forms of alopecia areata (circumscribed), anagen phase state without hair growth, traumatic alopecia, cicatrical alopecia, heat-induced alopecia, stress alopecia, alopecia induced by autoimmune disease (e.g. by discoid lupus erythematosus or chronic lupus erythematosus), disease-related alopecia (e.g. related with hypo- or hyperthyroidism, iron deficiency, zinc deficiency), drug-induced alopecia (e.g. induced by hormonal contraceptives, beta-adrenergic blockers, interferon, antineoplastic agents, allopurinol, bromocriptine); effects of: granulosarcoid, radiation therapy, poisonings (bismuth, arsenic, gold, boric acid, thallium).

26. The method for hair regrowth of claim 24, wherein the compositions are administered in a combination of the proper forms: gel, gel-cream.

27. The method of claim 24, wherein heparin or salts thereof formulated into preparations for topical hair regrowth is applied daily for 3 months, and after 3-month course on alternate days to the sites of hair loss.
